(19) United States
(12) Patent Application Publication
(10) Pub. No.: US 2012/0053414 A1
(43) Pub. Date: Mar. 1, 2012

(54) FLUID-TIGHT HOUSING DEVICE AND ENDOSCOPE

(75) Inventor: Haruhiko ARAI, Kanagawa (JP)

(73) Assignee: FUJIFILM CORPORATION, Tokyo (JP)

(21) Appl. No.: 13/198,572

(22) Filed: Aug. 4, 2011

(30) Foreign Application Priority Data
Aug. 25, 2010 (JP) 2010-188427

(51) Int. Cl. A61B 1/00

(52) U.S. Cl. 600/121

(57) ABSTRACT

A fluid-tight housing device for a section of a handle device of an endoscope includes a housing. A housing opening is formed in the housing, and includes plural side edges and plural corner portions disposed between the side edges in a curved manner. A cover is secured to the housing, for closing the housing opening. A seal packing is squeezed between the housing and the cover. A first sealing lip is formed to project from an end of the seal packing, entered in the housing opening upon securing the cover to the housing, pushed on an inner surface of the housing, for sealing a joint thereof in a fluid-tight manner. A projection is formed on the cover in association with at least one of the corner portions, to project toward the housing, for pressing the seal packing to increase tightness of the first sealing lip.

### BACKGROUND OF THE INVENTION

1. Field of the Invention

The present invention relates to a fluid-tight housing device and endoscope. More particularly, the present invention relates to a fluid-tight housing device and endoscope, in which a housing opening including side edges and corner portions can be closed reliably in a fluid-tight manner in a simplified structure.

2. Description Related to the Prior Art

There are devices in which electronic parts are contained and fluid must be prevented from entry, such as an underwater camera, water-proof watch, and the like. Each of those includes a fluid-tight housing device, which is used in an underwater condition, or may be washed frequently with water or other fluid. The fluid-tight housing device includes a housing and a cover. Relevant parts are contained in the fluid-tight housing device. An elastic seal packing of a loop shape is squeezed between the housing and the cover which are engaged with one another for firm connection. Thus, its inner chamber is closed in a fluid-tight manner. JP-A 2001-091996 discloses the fluid-tight housing device in which an O-ring is squeezed between the housing and the cover of a lens fitted photo film unit as the seal packing for a fluid-tight condition. A housing opening is defined in the housing, and has four corners with a curved shape at a small radius. If the O-ring of an annular shape is stretched in a quadrilateral shape, leakage of fluid occurs due to a smaller width at the corners of the quadrilateral shape. Note that a size of a small space between the housing and the cover for squeezing the O-ring is smaller at the corners than on the side edges.

There is another type of the fluid-tight housing device including a stationary housing, a movable part and an elastic seal packing disposed between the stationary housing and the movable part, for keeping the movable part movable and maintaining a fluid-tight condition without having the cover. JP-A 5-241227 discloses a zoom camera having the stationary housing, a movable lens barrel and the seal packing. The movable lens barrel is movable in the stationary housing. The seal packing is disposed between the stationary housing and the movable lens barrel, and has a V shape as viewed in a section, for the fluid-tight condition in a movable structure of the movable lens barrel. One edge of the V shape of the seal packing is fixed on the stationary housing. A second edge of the seal packing is deformed elastically, and contacts an outer side of the movable lens barrel with suitable pressure.

An inner chamber is defined in the housing of the fluid-tight housing device for containing electronic parts or the like. The inner chamber is closed by the cover fluid-tightly. A housing opening is disposed to open in the inner chamber in the housing. There are various ideas of shapes of the housing opening, such as a quadrilateral shape, polygonal shape and the like. An inner edge of the housing opening is constituted by plural side edges and plural corner portions between the side edges. The side edges are linear or slightly curved. The corner portions are angular or curved with a small radius. The seal packing in a shape similar to the housing opening is squeezed between the housing and the cover for keeping the inner chamber fluid-tight upon securing the cover to the housing. However, there is a problem in that a fluid-tight condition of the corner portions is lower remarkably than that of the side edges in the seal packing.

### SUMMARY OF THE INVENTION

In view of the foregoing problems, an object of the present invention is to provide a fluid-tight housing device and endoscope, in which a housing opening including side edges and corner portions can be closed reliably in a fluid-tight manner in a simplified structure.

In order to achieve the above and other objects and advantages of this invention, a fluid-tight housing device includes a housing. A housing opening is formed in the housing, includes plural side edges and plural corner portions disposed between the side edges in a curved manner. A cover is secured to the housing, for closing the housing opening. A seal packing is squeezed between the housing and the cover. A first sealing lip is formed to project from an end of the seal packing, entered in the housing opening upon securing the cover to the housing, pushed on an inner surface of the housing, for sealing a joint between the cover and the inner surface in a fluid-tight manner. A projection is formed on the cover in association with at least one of the corner portions, to project toward the inner surface, for pressing the seal packing to increase tightness of the first sealing lip on the inner surface.

Furthermore, a second sealing lip is formed to project from a second end of the seal packing different from the end with the first sealing lip. The first and second sealing lips project outwards substantially in a U shape as viewed in a section.

Furthermore, an end surface is formed on the housing around the housing opening, and opposed to the cover. A mating surface is formed on the cover, for receiving the end surface. A support surface is formed on the cover to extend erectly from the mating surface, opposed to the inner surface, for supporting the seal packing, the support surface having the projection.

The first sealing lip is disposed on an end side of the support surface from the second sealing lip in the housing, and has a projecting amount larger than a projecting amount of the second sealing lip.

The first sealing lip is pushed against the inner surface, and bent to direct a lip end toward the housing opening.

Furthermore, a receiving groove is formed in the support surface, for receiving entry of the seal packing internally.

The receiving groove is formed in each one of plural portions offset from the projection.

Furthermore, a cover edge portion is disposed in the cover, to extend in a housing longitudinal direction. An engaging groove is formed in the inner surface, for supporting the cover on the inner surface in a door form by receiving the cover edge portion before securing the cover, to position the cover with the housing opening.

The cover edge portion is constituted by an edge projection having a small thickness with respect to a direction of a thickness of the cover.

The housing includes a lateral wall, disposed to extend in the housing longitudinal direction, and having the engaging groove. An inclined wall is disposed opposite to the lateral wall, and inclined relative to the housing longitudinal direction to decrease a housing width.

Also, an endoscope including a section of a handle device and a fluid-tight housing device for containing the handle device is provided. The fluid-tight housing device includes a housing. A housing opening is formed in the housing, including plural side edges and plural corner portions disposed between the side edges in a curved manner. A cover is secured to the housing, for closing the housing opening. A seal packing is squeezed between the housing and the cover. A first sealing lip is formed to project from an end of the seal packing, entered in the housing opening upon securing the cover to the housing, pushed on an inner surface of the housing, for sealing a joint thereof in a fluid-tight manner. A projection is formed on the cover in association with at least one of the corner portions, to project toward the housing, for pressing the seal packing to increase tightness of the first sealing lip.

Furthermore, a cover edge portion is disposed in the cover, to extend in a housing longitudinal direction. An engaging groove is formed in the inner surface, for supporting the cover on the inner surface in a door form by receiving the cover edge portion before securing the cover, to position the cover with the housing opening.

Furthermore, an elongated tube is disposed to extend from the handle device on a distal side, for entry in a body cavity. The housing longitudinal direction is defined along an axis of the elongated tube.

The cover edge portion is constituted by an edge projection having a small thickness with respect to a direction of a thickness of the cover.

The housing includes a conduit receiving opening for receiving a fluid line extending from a fluid supply source to the handle device.

The housing includes a tube receiving opening, disposed opposite to the conduit receiving opening in a housing longitudinal direction, for receiving an elongated tube extending from the handle device.

The cover includes a cable opening for receiving an electrical cable extending from the handle device.

Therefore, a housing opening including side edges and corner portions can be closed reliably in a fluid-tight manner in a simplified structure, because of the use of the projection.

### BRIEF DESCRIPTION OF THE DRAWINGS

The above objects and advantages of the present invention will become more apparent from the following detailed description when read in connection with the accompanying drawings, in which:

FIG. 1 is a perspective view illustrating an endoscope system;

FIG. 2 is a perspective view illustrating a fluid-tight housing device;

FIG. 3 is an exploded perspective view illustrating the fluid-tight housing device;

FIG. 4 is a perspective view illustrating a housing;

FIG. 5 is a plan illustrating a cover;

FIG. 6 is a perspective view, partially broken illustrating a corner portion of the cover;

FIG. 7 is a cross section illustrating a seal packing;

FIG. 8 is a cross section illustrating a step of securing the cover to the housing;

FIG. 9 is a vertical section, partially broken illustrating the seal packing;

FIG. 10 is a vertical section, partially broken illustrating a side edge in the seal packing;

FIG. 11 is a vertical section, partially broken illustrating a corner portion in the seal packing;

FIG. 12 is a vertical section, partially broken illustrating a state of the seal packing with fluid pressure.

### DETAILED DESCRIPTION OF THE PREFERRED EMBODIMENT(S) OF THE PRESENT INVENTION

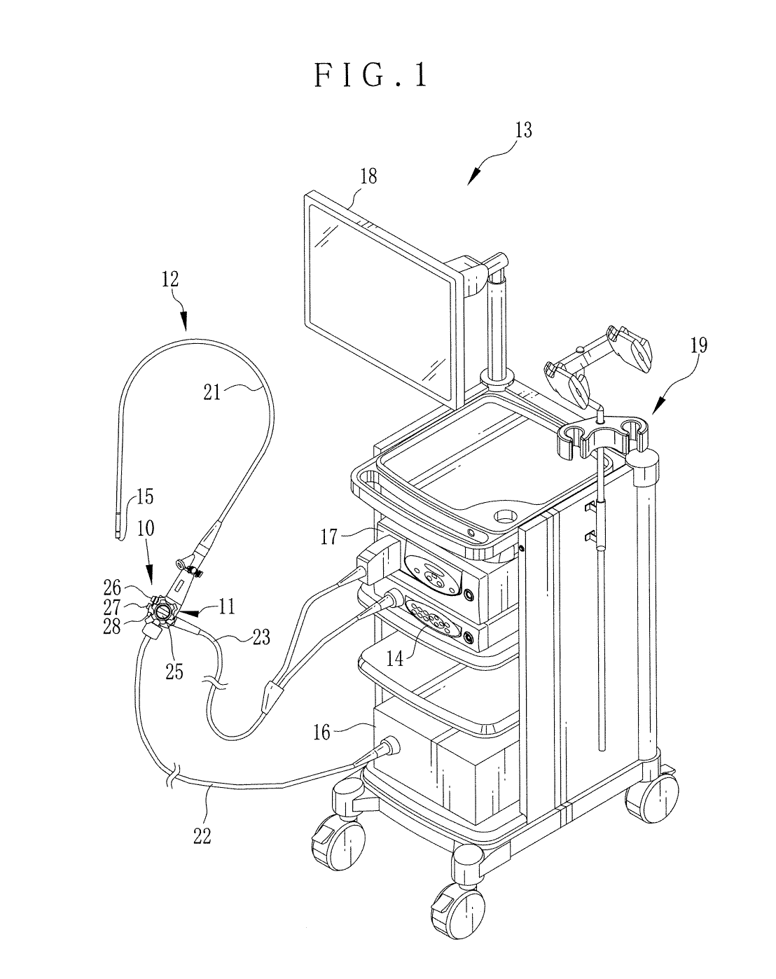

In FIG. 1, an endoscope 12 has a handle assembly, which includes a section of a handle device 10 and a fluid-tight housing device 11 or casing device for containing the handle device 10. The fluid-tight housing device 11 includes a manually operable mechanism and electronic parts as well-known in the field. The endoscope 12 after the use is washed up in a washing apparatus (not shown). All the elements of the endoscope 12 including the handle device 10 are shielded from fluid by a fluid-tight structure. An endoscope system 13 for diagnosis includes the endoscope 12, a processing apparatus 14, a fluid supply source 16, a light source apparatus 17, a monitor display panel 18 and a system cart 19. The endoscope 12 forms an image of a body cavity. The processing apparatus 14 controls the endoscope 12 electrically. A head assembly 15 of the endoscope 12 is cleaned by washing water from the fluid supply source 16. The light source apparatus 17 supplies light for illuminating the body cavity. The monitor display panel 18 displays the image of the body cavity. The system cart 19 supports those elements.

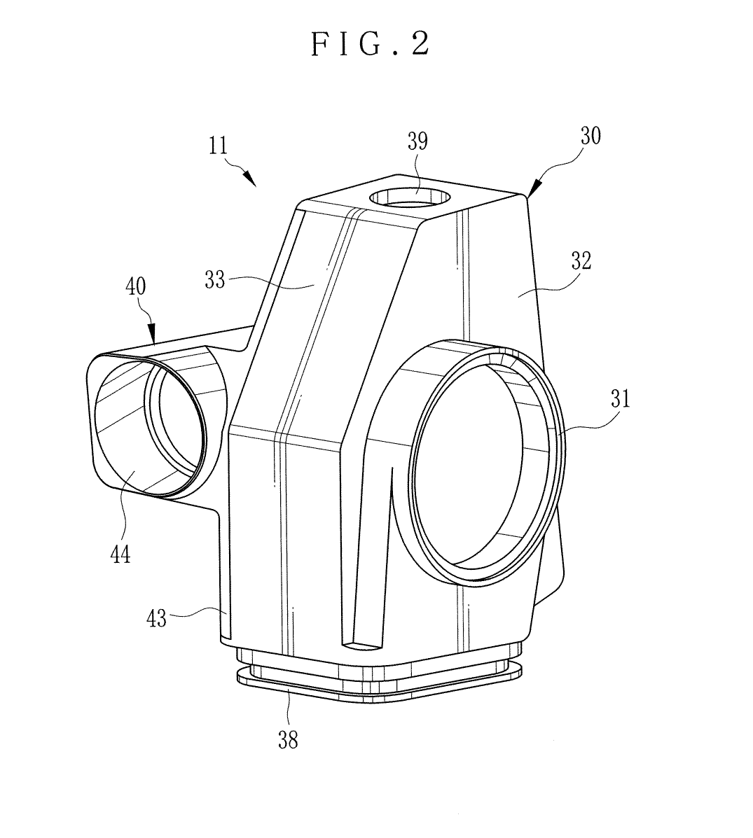

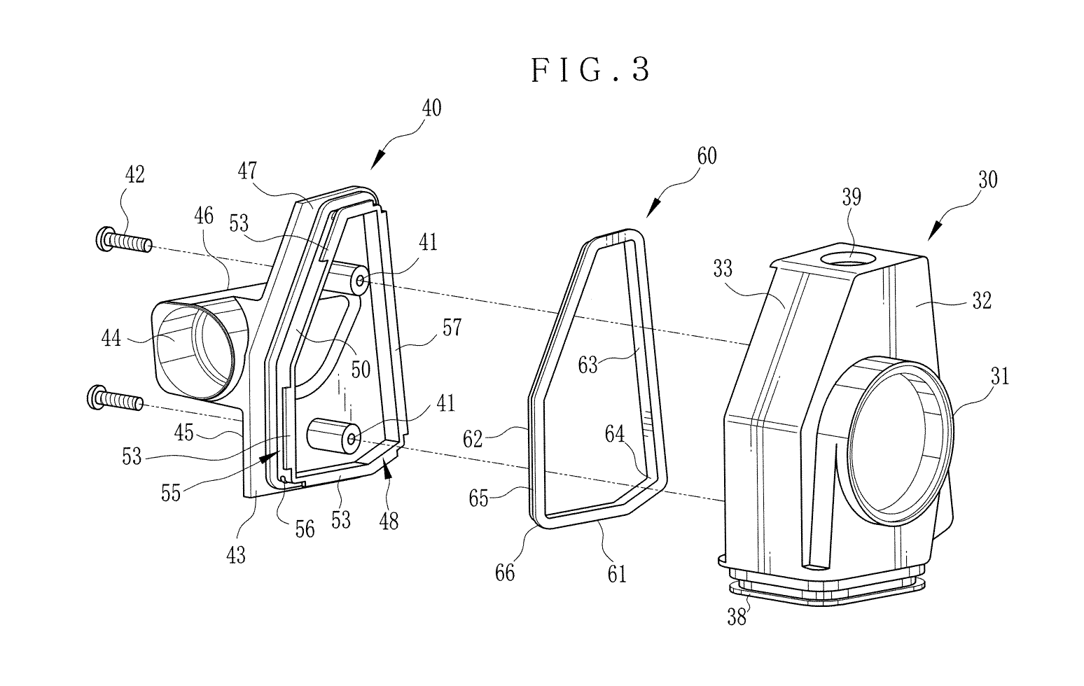

In FIGS. 2 and 3, the fluid-tight housing device 11 includes a housing 30 or casing, a cover 40, and a seal packing 60 with sealing lips. The seal packing 60 has resiliency, has a U shape as viewed in a cross section, and is directed to set the U portion in an outward direction. The cover 40 and the housing 30 with the seal packing 60 are assembled together. Then a screw 42 is entered in a screw hole 41 formed in the cover 40, and secures the cover 40 to the housing 30 firmly.

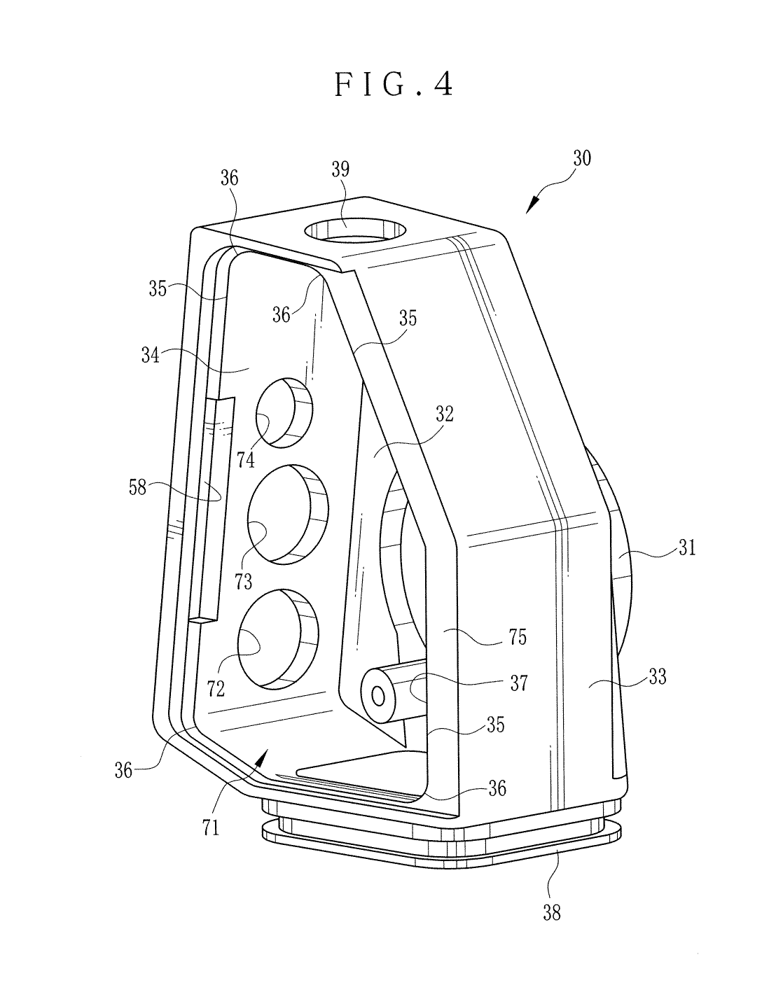

In FIGS. 2 and 4, the housing 30 includes a base plate 32, and a wall 33 formed to project from the base plate 32 in a box shape. A wheel support 31 is formed in the base plate 32. Steering wheels 25 are kept rotatable on the wheel support 31 for controlling a steering device on the head assembly 15. An inner chamber 71 is defined by the base plate 32 and the wall 33. The wall 33 has a housing opening 37, and an inner surface 34 extending from edges of the housing opening 37. The housing opening 37 is constituted by side edges 35 and corner portions 36, which are disposed between the side edges 35 and curved in an arcuate shape.

Various openings are formed in the wall 33, including a tube receiving opening 38 or tube coupling, a conduit receiving opening 39 and button openings 72, 73 and 74. An elongated tube 21 or guide tube of FIG. 1 is coupled with the tube receiving opening 38. A fluid line 22 or conduit extends from the fluid supply source 16 to draw fluid, and extends through the conduit receiving opening 39. An air/water button 26, a suction button 27 and an imaging button 28 of FIG. 1 are secured by entry in the button openings 72, 73 and 74 in a depressible manner by way of pushbuttons. An engaging groove 58 or second recess is formed in the inner surface 34, extends along one of the side edges 35 and is oriented vertically to the inner surface 34.

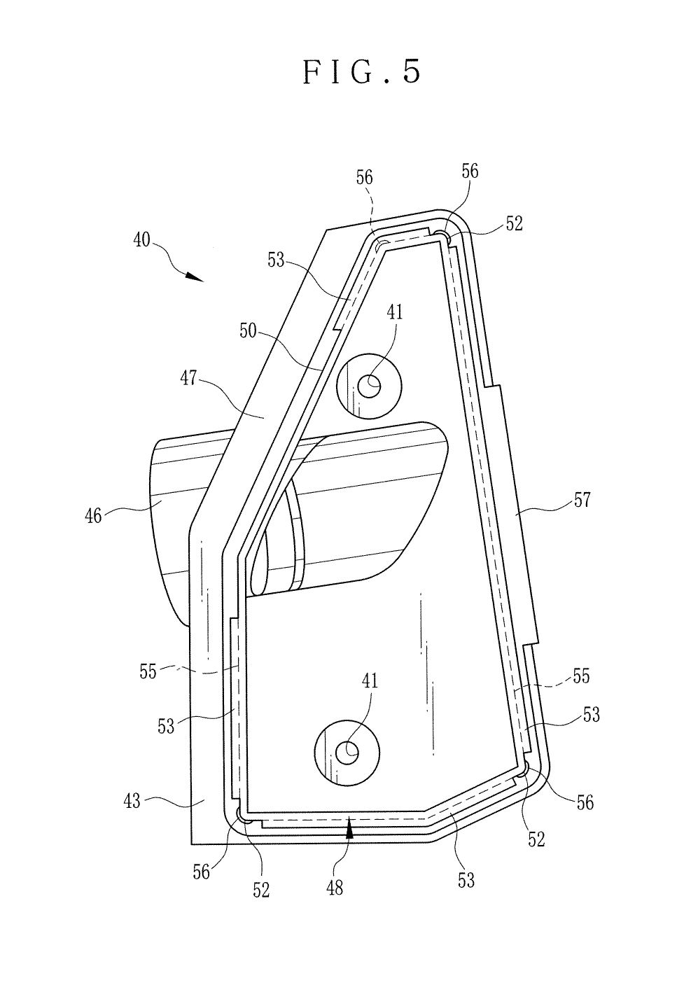

In FIGS. 3 and 5, the cover 40 includes a cover plate 43, a socket cover 46, and a contact support 48 for packing. An end surface 75 of the housing 30 is disposed around the housing opening 37. The cover plate 43 closes the inner chamber 71 by contact of a mating surface 47 with the end surface 75. The socket cover 46 is formed on a cover surface 45 of the cover plate 43. A cable opening 44 is open through the socket cover 46, and receives entry of a universal cable 23 for connection to the processing apparatus 14 and the light source apparatus 17. The contact support 48 has a support surface 50 for the seal packing. When the cover 40 closes the inner chamber 71, the support surface 50 enters the housing 30 and becomes opposed to the inner surface 34 with a clearance space. There is a corner portion 52 of the support surface 50. A first projection 56 is formed with the contact support 48 at the corner portion 52 and narrows the clearance space for tightening.

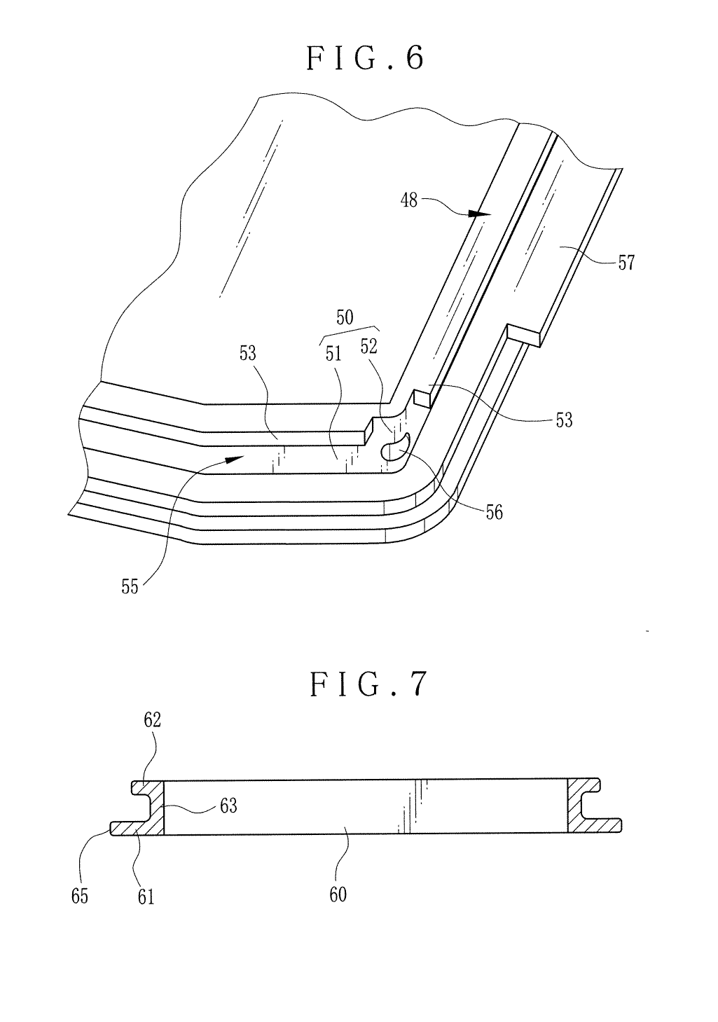

In FIG. 6, there are side edge portions 51 combined with the corner portion 52 to constitute the support surface 50. A first ridge 53 projects vertically from the support surface 50 at each of plural positions of the side edge portions 51. A receiving groove 55 or first recess is defined in a channel shape by the first ridge 53 for receiving entry of an inner band of the seal packing 60. An edge projection 57 or second ridge is formed with the cover plate 43, and engaged internally with the engaging groove 58 to operate as a pivot in a door form of the cover 40.

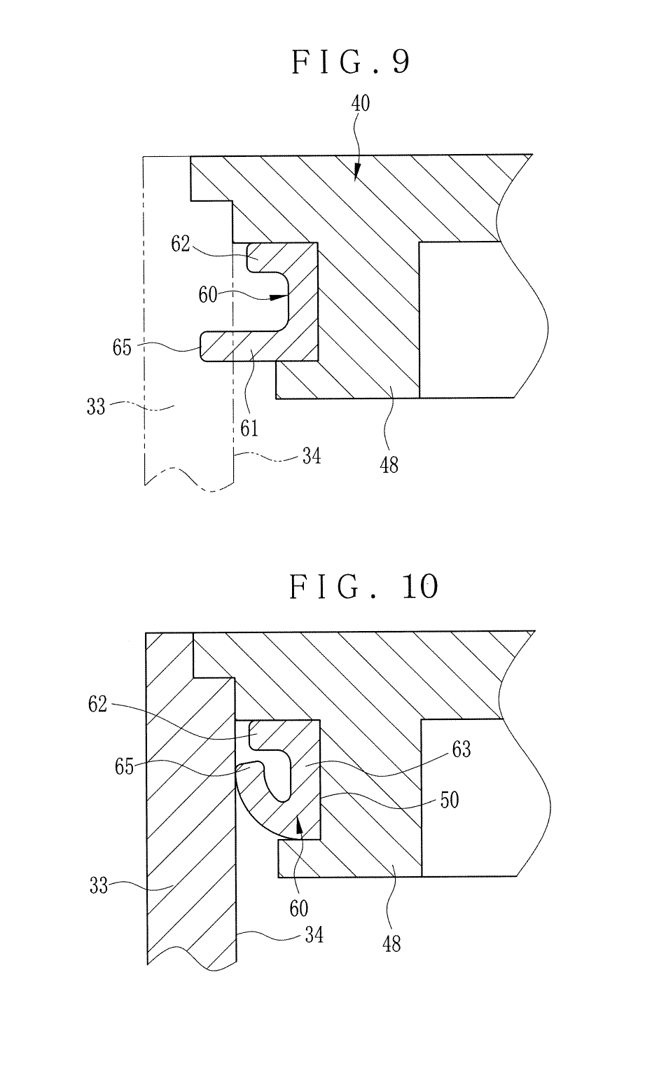

In FIGS. 3 and 7, the seal packing 60 includes a sealing band 63 or loop and first and second sealing lips 61 and 62. The sealing band 63 is fitted on the support surface 50 in tight contact internally. The first and second sealing lips 61 and 62 project in a U shape as viewed in a cross section, and extend in the circumferential direction continuously. The first sealing lip 61 has a larger size on the deeper side in the housing 30 than the second sealing lip 62 nearer to the housing opening 37. A lip end 65 of the first sealing lip 61 defines a profile, which is slightly larger than the housing opening 37 of the housing 30. A lip end of the second sealing lip 62 defines a profile, which is slightly smaller than the housing opening 37. See FIG. 9. When the cover 40 is set for closing, the lip end 65 of the first sealing lip 61 is pressed against the inner surface 34 at its full circumference. The inner chamber 71 is kept fluid-tight by tight contact between the first sealing lip 61 and the inner surface 34 and between the inside of the sealing band 63 and the support surface 50. See FIG. 10.

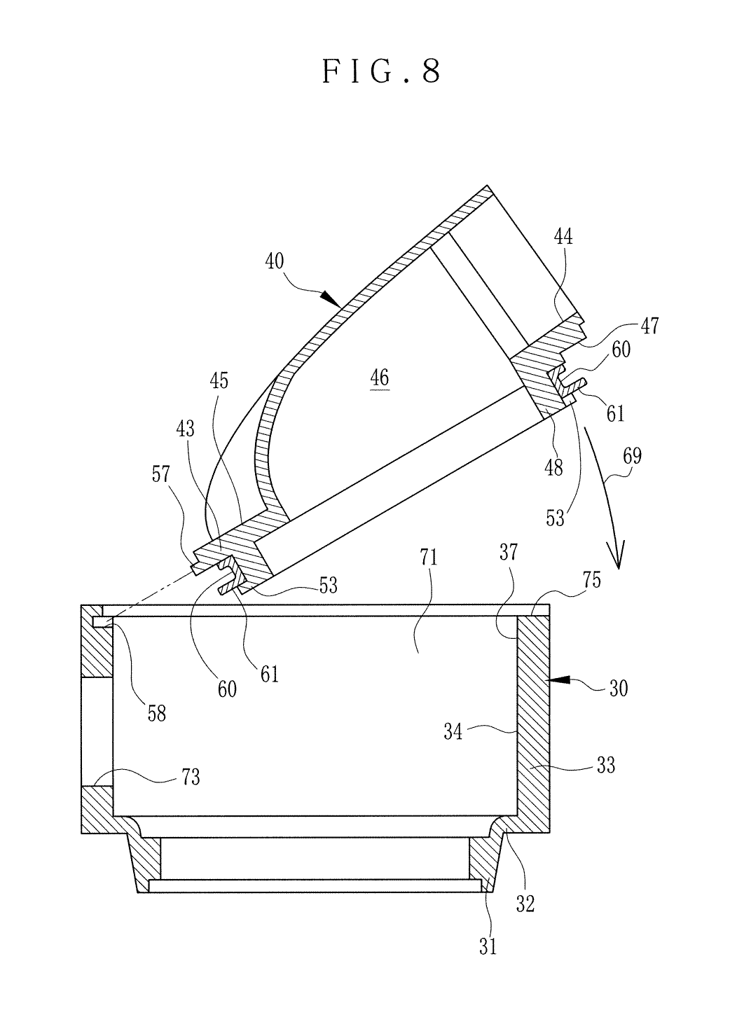

The operation of combining the cover 40 with the housing 30 and a fluid-tight condition of the seal packing 60 are described now. In FIG. 8, the seal packing 60 is fitted in the receiving groove 55 in the contact support 48 of the cover 40 by directing the first sealing lip 61 in an inward manner in the housing 30. After the edge projection 57 of the cover 40 is advanced into the engaging groove 58 of the housing 30, the cover 40 is rotated in an arrow direction 69 about the edge projection 57 to enter the contact support 48 in the inner chamber 71.

In FIGS. 9 and 10, the lip end 65 of the first sealing lip 61 has such a size as to reach a position of interference with the inner surface 34 of the housing 30. When the contact support 48 advances into the inner chamber 71, the first sealing lip 61 is bent down toward the second sealing lip 62. Return force of the first sealing lip 61 is exerted to the inner surface 34 and the support surface 50. The inner chamber 71 is kept fluid-tight by firmly pressing the lip end 65 on the inner surface 34 and the sealing band 63 on the support surface 50. There is a difference in the contact pressure between a first portion of the first sealing lip 61 associated with the side edges 35 of the inner surface 34 and a second portion associated with the corner portions 36 according to the return force of the first sealing lip 61. The second portion has the smaller contact pressure, and thus has a poorer fluid-tight performance.

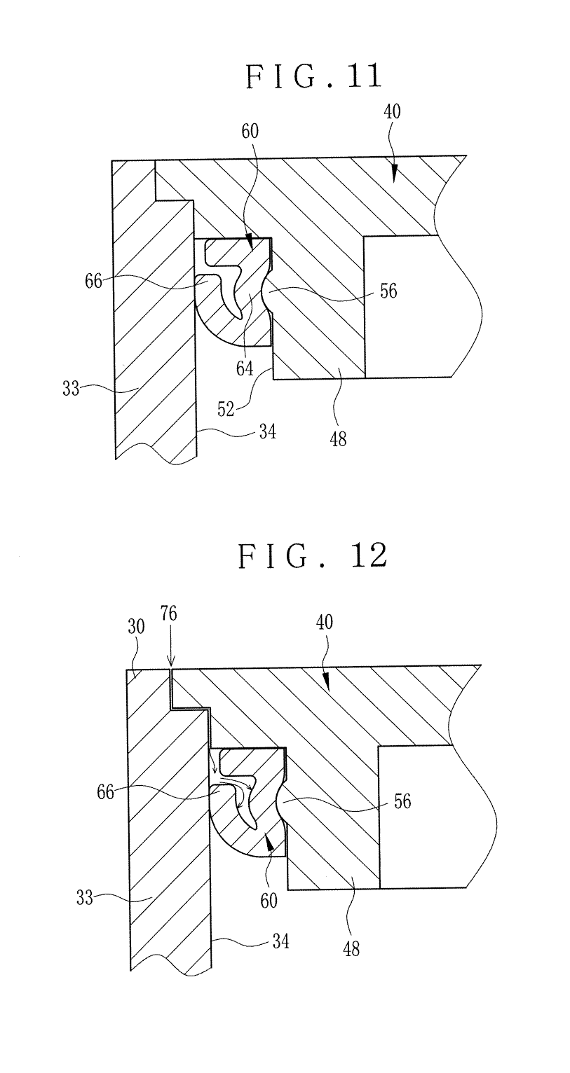

In FIG. 11, a corner portion 64 of the sealing band 63 in the seal packing 60 is pressed by the first projection 56 at the corner portion 52 of the support surface 50 toward the outside. A corner portion 66 of the lip end 65 of the first sealing lip 61 comes to have a higher pressure to press the inner surface 34. Thus, the fluid-tightness can be increased. In FIG. 12, a state of permeation of water along a mating surface 76 is illustrated between the housing 30 and the cover 40. The permeated water enters the inside of the seal packing 60 to spread the seal packing 60 outwards. Thus, the contact pressure between the seal packing 60 and the inner surface 34 and the first projection 56 can be higher to increase the fluid-tightness.

In the embodiment, the second sealing lip 62 does not protrude from a profile line of the housing opening 37. However, the second sealing lip 62 can have such a size as to protrude slightly from the profile line of the housing opening 37. When the cover 40 is present for closing, the lip end of the second sealing lip 62 is pressed by the inner surface 34 and bent toward the first sealing lip 61.

It is also preferable in the tube receiving opening 38, the conduit receiving opening 39 and the cable opening 44 to use a structure for preventing leakage of fluid after receiving a tube or conduit, for example an O-ring.

The fluid-tight housing device, although used in the endoscope, can be used for any of various instruments of a fluid-tight condition according to the invention.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A fluid-tight housing device comprising:
a housing;
a housing opening, formed in said housing, including plural side edges and plural corner portions disposed between said side edges in a curved manner;
a cover, secured to said housing, for closing said housing opening;
a seal packing squeezed between said housing and said cover;
a first sealing lip, formed to project from an end of said seal packing, entered in said housing opening upon securing said cover to said housing, pushed on an inner surface of said housing, for sealing a joint between said cover and said inner surface in a fluid-tight manner;
a projection, formed on said cover in association with at least one of said corner portions, to project toward said inner surface, for pressing said seal packing to increase tightness of said first sealing lip on said inner surface.

2. A fluid-tight housing device as defined in claim 1, further comprising a second sealing lip formed to project from a second end of said seal packing different from said end with said first sealing lip;
wherein said first and second sealing lips project outwards substantially in a U shape as viewed in a section.

3. A fluid-tight housing device as defined in claim 2, further comprising:
an end surface formed on said housing around said housing opening, and opposed to said cover;
a mating surface, formed on said cover, for receiving said end surface;
a support surface, formed on said cover erectly from said mating surface, opposed to said inner surface, for supporting said seal packing, said support surface having said projection.

4. A fluid-tight housing device as defined in claim 3, wherein said first sealing lip is disposed on an end side of said support surface from said second sealing lip in said housing, and has a projecting amount larger than a projecting amount of said second sealing lip.

5. A fluid-tight housing device as defined in claim 4, wherein said first sealing lip is pushed against said inner surface, and bent to direct a lip end toward said second sealing lip.

6. A fluid-tight housing device as defined in claim 5, further comprising a receiving groove, formed in said support surface, for receiving entry of said seal packing internally.

7. A fluid-tight housing device as defined in claim 6, wherein said receiving groove is formed in each one of plural portions offset from said projection.

8. A fluid-tight housing device as defined in claim 2, further comprising:
a cover edge portion, disposed in said cover, to extend in a housing longitudinal direction;
an engaging groove, formed in said inner surface, for supporting said cover on said inner surface in a pivotally movable form by receiving said cover edge portion before securing said cover.

9. A fluid-tight housing device as defined in claim 8, wherein said housing includes:
a lateral wall, disposed to extend in said housing longitudinal direction, and having said engaging groove;
an inclined wall, disposed opposite to said lateral wall, and inclined relative to said housing longitudinal direction to decrease a housing width.

10. An endoscope including a fluid-tight housing device for containing a section of a handle device, comprising:
said fluid-tight housing device including:
a housing;
a housing opening, formed in said housing, including plural side edges and plural corner portions disposed between said side edges in a curved manner;
a cover, secured to said housing, for closing said housing opening;
a seal packing squeezed between said housing and said cover;
a first sealing lip, formed to project from an end of said seal packing, entered in said housing opening upon securing said cover to said housing, pushed on an inner surface of said housing, for sealing a joint between said cover and said inner surface in a fluid-tight manner;
a projection, formed on said cover in association with at least one of said corner portions, to project toward said housing, for pressing said seal packing to increase tightness of said first sealing lip on said inner surface.

11. An endoscope as defined in claim 10, further comprising a second sealing lip formed to project from a second end of said seal packing different from said end with said first sealing lip;
wherein said first and second sealing lips project outwards substantially in a U shape as viewed in a section.

12. An endoscope as defined in claim 11, further comprising:
an end surface formed on said housing around said housing opening, and opposed to said cover;
a mating surface, formed on said cover, for receiving said end surface;
a support surface, formed on said cover erectly from said mating surface, opposed to said inner surface, for supporting said seal packing, said support surface having said projection.

13. An endoscope as defined in claim 12, wherein said first sealing lip is disposed on an end side of said support surface from said second sealing lip in said housing, and has a projecting amount larger than a projecting amount of said second sealing lip.

14. An endoscope as defined in claim 11, wherein said housing includes a conduit receiving opening for receiving passage of a fluid line connected with a fluid supply source.

15. An endoscope as defined in claim 14, wherein said housing includes a tube receiving opening, disposed opposite to said conduit receiving opening in a housing longitudinal direction, for receiving passage of an elongated tube for entry in a body cavity.

16. An endoscope as defined in claim 11, wherein said cover includes a cable opening for receiving passage of an electrical cable.
